(19) United States
(12) Patent Application Publication
(10) Pub. No.: US 2016/0371721 A1
(43) Pub. Date: Dec. 22, 2016

(54) METHODS AND SYSTEMS FOR MONITORING BRAIN ACTIVITY OF A VIEWER DURING AN ADVERTISEMENT

(71) Applicant: Rovi Guides, Inc., Santa Clara, CA (US)

(72) Inventors: Robert Bogdon, San Diego, CA (US); Walter R. Klappert, Los Angeles, CA (US)

(21) Appl. No.: 14/742,860

(22) Filed: Jun. 18, 2015

(51) Int. Cl. G06Q 30/02; A61B 5/048; A61B 5/0488; A61B 5/0484

(52) U.S. Cl. CPC G06Q 30/0242; A61B 5/04842; A61B 5/04845; A61B 5/048; A61B 5/0488; A61B 2503/12

(57) ABSTRACT

Methods and systems are disclosed herein for determining whether a viewer watched an advertisement. In some embodiments, control circuitry generates for display an advertisement including a stimulus. For example, the stimulus may be incorporated into the advertisement by a content provider in order to trigger a region of the viewer's brain. The control circuitry monitors brain activity in the region of the viewer's brain during the advertisement. The control circuitry determines a brain state associated with the region based on the brain activity of the viewer during the advertisement. The control circuitry compares the brain state to a threshold range associated with the stimulus included in the advertisement. In response to determining that the brain state matches the threshold range, the control circuitry stores in a database an indication that the viewer watched the advertisement.

### BACKGROUND

In conventional systems, an advertiser may estimate an audience size of viewers for their advertisement based on a number of viewer televisions on which their advertisement is displayed. However, determining whether the advertisement was displayed on a viewer's television may not guarantee that the viewer watched the advertisement. For example, the viewer may be present near the television but may look away during presentation of the advertisement on the viewer's television. In another example, the viewer may walk away from the television, e.g., to get a snack, during presentation of the advertisement on the viewer's television.

### SUMMARY

Accordingly, systems and methods are disclosed herein for monitoring brain activity of a viewer (hereinafter interchangeably referred to as a user) during an advertisement to determine whether the viewer watched the advertisement. The systems and methods disclosed herein may apply in a similar manner to product placements in television programs and other suitable advertising media.

In some aspects, the viewer may wear a user device associated with monitoring brain activity. When an advertisement including a stimulus, e.g., a strobe of light, is displayed on the viewer's television or another suitable display device, a region of the viewer's brain may be triggered. The user device may detect the triggering of the region of the viewer's brain, e.g., the occipital lobe, in response to the stimulus. The user device or another suitable device may compare brain activity for the region of the viewer's brain to a threshold range associated with the stimulus to determine whether the viewer watched the advertisement.

In some embodiments, the user device monitors brain activity of the viewer and determines a brain state of the viewer based on the brain activity. The brain state (e.g., the current frequency range of voltage fluctuations in the brain and/or electrical activity of muscles near the brain at rest and during contraction) may correspond to a region of the viewer's brain during presentation of the advertisement. The user device or another suitable device may select a threshold range (e.g., a target frequency range of voltage fluctuations in the brain and/or electrical activity of muscles near the brain at rest and during contraction) that corresponds to the stimulus included in the advertisement. The user device may compare the brain state to the threshold range to determine whether there is a match (e.g., indicative of whether or not a viewer watched the advertisement).

To determine the advertisement corresponding to a brain state, the user device or another suitable device may cross-reference data associated with the brain state of the viewer (e.g., the frequency range and/or electrical activity of the muscles near the brain) with a database associated with data related to brain states and corresponding advertisements. For example, based on the cross-reference, the user device may determine that the data associated with the brain state of the viewer indicates that the viewer watched a COKE advertisement, a FORD advertisement, or another suitable advertisement. The user device may then select a threshold range corresponding to the presented advertisement based on a time when the advertisement was displayed, a user input indicating the advertisement, metadata associated with the advertisement, or other suitable information. For example, a COKE advertisement may correspond to a particular threshold range while a FORD advertisement may correspond to a different threshold range.

In some embodiments, the user device incorporates and/or has access to an electroencephalogram unit (“EEG”) indicating a frequency range of voltage fluctuations in the brain activity of a viewer and/or an electromyogram unit (“EMG”) indicating electrical activity of muscles near a brain of the viewer at rest and during contraction. For example, an EMG may indicate (by detecting the electrical activity of muscles associated with the blinking of the eyes of a viewer) that a viewer is currently blinking his/her eyes. Furthermore, the EEG and/or the EMG may be incorporated into a battery-powered mobile headset (e.g., styled as traditional headphones, hats/helmets, glasses, etc.) upon which a media guidance application is implemented and/or in communication with. The monitoring may be initiated in response to a user input entered on the user device (e.g., turning the user device on), in response to detecting a change (or lack thereof) in brain activity, and/or based on a predetermined schedule (e.g., when an advertisement is scheduled to be presented).

In some aspects, the methods and systems disclosed herein provide for a method for determining whether a viewer watched an advertisement. The method includes generating for display an advertisement including a stimulus. The stimulus is incorporated into the advertisement by a content provider, e.g., an advertiser, in order to trigger a region of the viewer's brain. The method further includes monitoring brain activity in the region of the viewer's brain during the advertisement. The method further includes determining a brain state associated with the region based on the brain activity of the viewer during the advertisement. The method further includes comparing the brain state to a threshold range associated with the stimulus included in the advertisement. The method further includes storing in a database an indication that the viewer watched the advertisement in response to determining that the brain state matches the threshold range.

For example, a user device monitoring the viewer's brain during the advertisement may detect a state of the brain activity associated with the occipital lobe of the viewer. The advertisement may include a stimulus of flashing lights that triggered the occipital lobe of the viewer. The user device or another suitable device may compare the brain state of the viewer to a threshold range associated with the stimulus included in the advertisement to determine whether the viewer watched the advertisement. In response to determining that the brain state of the viewer matches the threshold range for the stimulus included in the advertisement, the user device may return a message indicating a match and that the viewer watched the advertisement. In response to determining that the brain state of the viewer does not match the threshold range for the stimulus included in the advertisement, the user device may return a message indicating no match and that the viewer did not watch the advertisement.

In some embodiments, the stimulus in the advertisement includes one or more of a visual stimulus, an audio stimulus, an audiovisual stimulus, a video stimulus, a graphical stimulus, a textual stimulus, or another suitable stimulus. For example, the content provider, e.g., the advertiser, may include a graphical stimulus, such as flashing lights, to trigger the occipital lobe of the viewer since it is associated with the viewer's vision. In another example, the content provider, e.g., the advertiser, may include a textual stimulus to trigger the parietal lobe of the viewer since it is associated with the viewer's reading ability. In yet another example, the content provider, e.g., the advertiser, may include an audio stimulus to trigger the temporal lobe of the viewer since it is associated with the viewer's hearing. The content provider may include any combination of one or more stimuli to trigger various regions of the viewer's brain. The regions triggered by the combination of stimuli may indicate that the viewer watched a particular advertisement. For example, a visual trigger of flashing lights and an audio trigger of a tonal sound may be uniquely associated with a particular advertisement. Detection of corresponding triggers in the occipital and temporal lobes of the viewer may indicate the viewer watched the particular advertisement.

In some embodiments, monitoring the brain activity of the viewer includes receiving data from an electroencephalogram unit indicating a frequency range of the brain activity of the viewer or receiving data from an electromyogram unit indicating electrical activity of muscles near the brain of the viewer at rest and during contraction. Depending on the type of monitoring used, the threshold range for the stimulus may be associated with the frequency range of the brain activity or the electrical activity of muscles near the brain. The threshold range may be stored in a database and, when required, retrieved by a user device monitoring the brain activity of the viewer. In some embodiments, the electroencephalogram data and the electromyogram data are received from separate devices. In some embodiments, the electroencephalogram data and the electromyogram data are received from the same device.

In some embodiments, the region of the brain includes an occipital lobe, a parietal lobe, a temporal lobe, or a frontal lobe. The content provider, e.g., the advertiser, may include different stimuli to trigger the different regions of the viewer's brain. For example, the content provider may include a visual stimulus in the advertisement to trigger the occipital lobe of the viewer's brain. This is because the occipital lobe is associated with the viewer's vision. In another example, the content provider may include an audio stimulus in the advertisement to trigger the temporal lobe of the viewer's brain. This is because the temporal lobe is associated with the viewer's hearing. In yet another example, the content provider may include a textual stimulus in the advertisement to trigger the parietal lobe of the viewer's brain. This is because the parietal lobe is associated with the viewer's reading ability. In yet another example, the content provider may include an emotional or mood-related stimulus in the advertisement to trigger the frontal lobe of the viewer's brain. This is because the frontal lobe is associated with the viewer's emotions or mood.

In some embodiments, the threshold range for the stimulus included in the advertisement is associated with a response from the region of the brain triggered by the included stimulus. Depending on the type of monitoring used, the threshold range for the stimulus may be associated with the frequency range of the brain activity or the electrical activity of muscles near the brain. The threshold range may be stored in a database and, when required, retrieved by a user device monitoring the brain activity of the viewer. A user device monitoring the viewer's brain activity may select a threshold range corresponding to a presented advertisement based on a time when the advertisement was displayed, a user input indicating the advertisement, metadata associated with the advertisement, or other suitable information. For example, a COKE advertisement may correspond to a particular threshold range while a FORD advertisement may correspond to a different threshold range.

In some embodiments, the stimulus includes a strobe of light, the region includes an occipital lobe of the viewer, and the threshold range is associated with a response from the occipital lobe in response to the strobe of light displayed during the advertisement. In this exemplary embodiment, the content provider, e.g., the advertiser, includes a strobe of light in the advertisement. The strobe of light triggers the occipital lobe of the viewer's brain since the occipital lobe is associated with the viewer's vision. The user device monitoring the viewer's brain activity or another suitable device retrieves a threshold range from the database for comparing against the viewer's brain state based on the brain activity. The threshold range is associated with a response from the occipital lobe in response to the strobe of light displayed during the advertisement. In response to determining that the brain state of the viewer matches the threshold range for the stimulus included in the advertisement, the user device may return a message indicating a match and that the viewer watched the advertisement. In response to determining that the brain state of the viewer does not match the threshold range for the stimulus included in the advertisement, the user device may return a message indicating no match and that the viewer did not watch the advertisement.

In some embodiments, the method further includes cross-referencing with a database to determine the advertisement the viewer has watched. After a user device monitoring the viewer's brain activity detects a trigger in a region of the viewer's brain, the user device or another suitable device may determine which advertisement the viewer watched. In one example, the database includes reference brain states associated with multiple advertisements. Cross-referencing with the database to determine which advertisement the viewer has watched includes determining which reference brain state matches the viewer's brain state. For example, a reference brain state invoked by a visual trigger of flashing lights and an audio trigger of a tonal sound may be uniquely associated with a particular advertisement. Detection of corresponding triggers in the occipital and temporal lobes of the viewer may indicate the viewer watched the particular advertisement. In another example, the database includes reference metadata associated with multiple advertisements. Cross-referencing with the database to determine which advertisement the viewer has watched includes determining which reference metadata matches the metadata for the advertisement presented to the viewer.

In some embodiments, the method further includes updating a count for the number of instances the viewer has watched the advertisement. For example, after determining which advertisement the viewer has watched, a user device monitoring the viewer's brain activity or another suitable device may send an indication to the database to update the number of times the viewer has watched the advertisement. The indication may include additional information regarding the advertisement, such as presentation time, presentation channel, and other suitable information. The content provider may utilize the information from the database to improve their future advertising efforts.

In some aspects, the systems and methods described herein include a system or an apparatus for determining whether a viewer watched an advertisement.

It should be noted, the systems, methods, apparatuses, and/or aspects described above may be applied to, or used in accordance with, other systems, methods, apparatuses, and/or aspects.

### BRIEF DESCRIPTION OF THE DRAWINGS

The above and other objects and advantages of the disclosure will be apparent upon consideration of the following detailed description, taken in conjunction with the accompanying drawings, in which like reference characters refer to like parts throughout, and in which:

FIG. 1 shows an illustrative media guidance application for selecting media assets in accordance with some embodiments of the disclosure;

FIG. 2 shows an illustrative media guidance application that may be used to adjust user settings in accordance with some embodiments of the disclosure;

FIG. 3 is a block diagram of an illustrative user equipment device in accordance with some embodiments of the disclosure;

FIG. 4 is a block diagram of an illustrative media system in accordance with some embodiments of the disclosure;

FIG. 5 shows an illustrative representation of monitoring brain activity of a viewer during an advertisement to determine whether the viewer watched the advertisement in accordance with some embodiments of the disclosure;

FIG. 6 shows a representation of a user and regions of the brain of the user associated with monitoring brain activity in accordance with some embodiments of the disclosure;

FIG. 7 shows multiple user devices that may be associated with monitoring brain activity in accordance with some embodiments of the disclosure;

FIG. 8 is a flow-chart of illustrative steps involved in monitoring brain activity of a viewer during an advertisement to determine whether the viewer watched the advertisement in accordance with some embodiments of the disclosure; and

FIG. 9 is a flow-chart of illustrative steps involved in comparing a viewer's brain state to a threshold range associated with a stimulus included in an advertisement in accordance with some embodiments of the disclosure.

### DETAILED DESCRIPTION OF DRAWINGS

The amount of content available to users in any given content delivery system can be substantial. Consequently, many users desire a form of media guidance through an interface that allows users to efficiently navigate content selections and easily identify content that they may desire. An application that provides such guidance is referred to herein as an interactive media guidance application or, sometimes, a media guidance application or a guidance application.

Interactive media guidance applications may take various forms depending on the content for which they provide guidance. One typical type of media guidance application is an interactive television program guide. Interactive television program guides (sometimes referred to as electronic program guides) are well-known guidance applications that, among other things, allow users to navigate among and locate many types of content or media assets. Interactive media guidance applications may generate graphical user interface screens that enable a user to navigate among, locate and select content. As referred to herein, the terms “media asset” and “content” should be understood to mean an electronically consumable user asset, such as television programming, as well as pay-per-view programs, on-demand programs (as in video-on-demand (VOD) systems), Internet content (e.g., streaming content, downloadable content, Webcasts, etc.), video clips, audio, content information, pictures, rotating images, documents, playlists, websites, articles, books, electronic books, blogs, advertisements, chat sessions, social media, applications, games, and/or any other media or multimedia and/or combination of the same. Guidance applications also allow users to navigate among and locate content. As referred to herein, the term “multimedia” should be understood to mean content that utilizes at least two different content forms described above, for example, text, audio, images, video, or interactivity content forms. Content may be recorded, played, displayed or accessed by user equipment devices, but can also be part of a live performance.

With the advent of the Internet, mobile computing, and high-speed wireless networks, users are accessing media on user equipment devices which they traditionally did not use. As referred to herein, the phrase “user equipment device,” “user equipment,” “user device,” “electronic device,” “electronic equipment,” “media equipment device,” or “media device” should be understood to mean any device for accessing the content described above, such as a television, a Smart TV, a set-top box, an integrated receiver decoder (IRD) for handling satellite television, a digital storage device, a digital media receiver (DMR), a digital media adapter (DMA), a streaming media device, a DVD player, a DVD recorder, a connected DVD, a local media server, a BLU-RAY player, a BLU-RAY recorder, a personal computer (PC), a laptop computer, a tablet computer, a WebTV box, a personal computer television (PC/TV), a PC media server, a PC media center, a hand-held computer, a stationary telephone, a personal digital assistant (PDA), a mobile telephone, a portable video player, a portable music player, a portable gaming machine, a smart phone, or any other television equipment, computing equipment, or wireless device, and/or combination of the same. In some embodiments, the user equipment device may have a front facing screen and a rear facing screen, multiple front screens, or multiple angled screens. In some embodiments, the user equipment device may have a front facing camera and/or a rear facing camera. On these user equipment devices, users may be able to navigate among and locate the same content available through a television. Consequently, media guidance may be available on these devices, as well. The guidance provided may be for content available only through a television, for content available only through one or more of other types of user equipment devices, or for content available both through a television and one or more of the other types of user equipment devices. The media guidance applications may be provided as on-line applications (i.e., provided on a web-site), or as stand-alone applications or clients on user equipment devices. Various devices and platforms that may implement media guidance applications are described in more detail below.

In some embodiments, a user device may be configured to monitor brain activity upon which various media guidance application operations and features may be based. In some embodiments, the user device may incorporate and/or have access to an electroencephalogram unit (“EEG”). An EEG measures electrical activity associated with a brain of a user. For example, an EEG may measure voltage fluctuations and/or the frequency or frequency range of voltage fluctuations generated by the brain of a user.

For example, an EEG may describe rhythmic brain activity. Rhythmic activity (e.g., activity associated with neural oscillation) also known as brain waves may be described in terms of frequency bands or frequency ranges. For example, a delta band includes a frequency range of up to about 4 Hz with a typical amplitude of 20-200 microvolts. Delta bands are, in some circumstances, associated with a sleeping state of a user. Theta bands include a frequency range of 4 to 8 Hz with a typical amplitude of 10 microvolts. Theta bands are, in some circumstances, associated with drowsiness. Alpha bands include a frequency range of 8 to 13 Hz with a typical amplitude of 20-200 microvolts. Alpha bands are, in some circumstances, associated with a relaxed state and/or the blinking of a user's eyes. Beta bands include frequencies of 13 to 30 Hz with a typical amplitude of 5-10 microvolts. Beta bands are, in some circumstances, associated with alertness, concentration, and/or anxiety. Gamma bands include a frequency range of 30 to 100 Hz and may have various amplitudes. Gamma bands are, in some circumstances, associated with combinations of senses of a user (e.g., sight, smell, sound, touch, taste) and/or short term memory. Frequency bands and frequency ranges as well as the symmetry of these bands and ranges across the brain of a user are also associated with various moods, which is discussed in detail in Rybak, “Frontal Alpha Power Asymmetry in Aggressive Children and Adolescents With Mood and Disruptive Behavior Disorders,” Clinical EEL and Neuroscience, Vol. 3, 2006, which is hereby incorporated by reference herein in its entirety.

Additional discussion about the use of EEG's to detect a level of attention, engagement, frustration, anxiety, emotional state, and comprehension are discussed in detail in Wyczesany, Miroslaw et al., “Subjective mood estimation co-varies with spectral power EEG characteristics,” Department of Psychophysiology, Jagiellonian University, Krakow, Poland, Acta Neurobiol Exp, 68: 180-192, 2008, Tan, Bao Hong, “Using a Low-cost EEG Sensor to Detect Mental States, CMU-CS-12-134, School of Computer Science, Carnegie Mellon University, August 2012, Hamadicharef et al., “Learning EEG-based Spectral-Spatial Patterns for Attention Level Measurement,” Institute for Infocomm Research, 2009, Bos, Danny Oude, “EEG-based Emotion Recognition, The Influence of Visual and Auditory Stimuli,” Department of Computer Science, University of Twente, 2006, and Pradeep et al., U.S. Pat. No. 8,392,250, issued Mar. 5, 2013, which are hereby incorporated by reference herein in their entirety.

In some embodiments, the user device incorporates and/or has access to an electromyogram unit (“EMG”). An EMG measures the electrical activity of muscles at rest and during contraction. The use of EMG and EEG for providing biofeedback is discussed in detail in Frank et al., “Biofeedback in medicine: who, when, why and how?” Ment. Health Fam. Med., June 2010, and Wartena et al., U.S. Patent Application Publication No. 2012/0029322, filed Mar. 24, 2010, which is hereby incorporated by reference herein in its entirety. In some embodiments, the user device may include additional components for detecting brain activity, moods, and attentiveness of a user as discussed in detail in Lee et al., U.S. Pat. No. 8,332,883, issued Dec. 11, 2012, and Bill, U.S. Pat. No. 8,373,768, issued Feb. 12, 2013, which are hereby incorporated by reference herein in their entirety.

In some embodiments, a user device may also distinguish between the different areas of the brain and the different functions of each area of the brain. For example, the frontal lobes are typically associated with planning, problem-solving, voluntary motor control, cognition, intelligence, attention, language processing and comprehension, and various emotions. The parietal lobe is typically associated with perception and integration of somatosensory information (e.g., touch, pressure, temperature, and pain) visuospatial processing, spatial attention, spatial mapping, and number representation. The occipital lobe is typically associated with vision, including color, orientation, and motion. The temporal lobe is typically associated with recognition, perception, hearing, smell, and memory. The regions and functions of the brain, in particular their effect on attention and emotion are discussed in detail in Yamasaki et al., “Dissociable prefrontal brain systems for attention and emotion,” Proceedings of the National Academy of Sciences, vol. 99, no. 17, 2002, which is hereby incorporated by reference in its entirety.

In some embodiments, a user device may be configured as a headset. As used herein a “headset” refers to any device or article worn or affixed to a user for monitoring brain activity. For example, a user device for monitoring brain activity may be fashioned as a pair of headphones, a hat, a helmet, a pair of glasses, and/or other configuration for use by a user. In some embodiments, a headset may be powered by a local energy storage device (e.g., a battery). For example, in some embodiments, a headset may be rechargeable and/or include replaceable energy storage devices.

In some embodiments, a media guidance application (or a user device upon which the media guidance application is implemented) may detect and/or monitor brain activity of a user. In some embodiments, the media guidance application may determine whether or not the brain activity of a user corresponds to a threshold range. As referred to herein, a “threshold range” refers to a frequency range and/or amplitude of brain activity that defines the boundaries of a brain state. For example, a threshold range may be defined as a particular frequency range (in Hz) associated with a brain state of a user, may be defined as frequency bands associated with a brain state of a user, and/or may be defined according to any other measurement that describes the current, preferred, past, and/or future brain state of a user. In some embodiments, a threshold range may account for any transient variations and amplitudes in brain state. For example, a threshold range may be defined as an average amplitude, frequency, frequency range, and/or frequency band over a particular period of time. In addition, a threshold range may refer to a composite range that includes one or more amplitudes and/or frequencies associated with one or more waves. For example, in some embodiments, a particular brain state may correspond to brain activity corresponding to theta bands with a first amplitude and delta bands at a second amplitude.

It should also be noted that in some embodiments, a threshold range may itself include one or more threshold ranges. For example, a threshold range associated with one brain state (e.g., a user being awake) may itself include numerous other threshold ranges (e.g., a mood of the user, an attentiveness level of the user, etc.).

As referred to herein, a “brain state” refers to a qualitative assessment of the mood, level of anxiety, level of attentiveness, level of comprehension, level of proficiency associated with one or more functions (e.g., reading text on a screen, hearing audio, etc.) of a user, and/or a combination thereof associated with the brain activity of the user. A brain state can be quantified as corresponding to a particular threshold range, and different brain states may be compared based on their corresponding threshold ranges.

Brain states may be identified by a user device (e.g., upon which a media guidance application is implemented) that incorporates and/or have access to a device for monitoring brain waves (e.g., an EEG, EMG, and/or any other device discussed herein). The media guidance application may monitor the brain activity (e.g., brain waves) of a user and determine a brain state of the user based on the brain activity. The brain state (e.g., the current frequency range of voltage fluctuations in the brain, electrical activity of muscles near the brain at rest and during contraction, and/or threshold range) may correspond to a trigger in a region of the user's brain. The media guidance application may select a threshold range (e.g., a target frequency range of voltage fluctuations in the brain, electrical activity of muscles near the brain at rest and during contraction, and/or threshold range) that corresponds to a stimulus that triggered the region of the user's brain. The media guidance application may compare the brain state to the threshold range (e.g., compare the frequency range of voltage fluctuations in the brain, electrical activity of muscles near the brain at rest and during contraction, and/or threshold range associated with the brain state to the frequency range of voltage fluctuations in the brain, electrical activity of muscles near the brain at rest and during contraction, and/or threshold range associated with the threshold range) to determine whether the two measurements correspond (e.g., indicate the same frequency range of voltage fluctuations in the brain, electrical activity of muscles near the brain at rest and during contraction, and/or threshold range).

The media guidance application may perform numerous operations for the user. As referred to herein, a “media guidance application operation” refers to any operation corresponding to providing, receiving, and generating media guidance data for consumption by a user. For example, media guidance application operations include displaying media guidance data, providing options to navigate, select, and edit media guidance data or content (e.g., a media asset) associated with media guidance data, and/or manipulating a device used to access (e.g., a display device), retrieve (e.g., a server), and/or associate media guidance data with a user (e.g., a user device for monitoring brain activity). One of the operations of the media guidance application is to provide media guidance data to users. As referred to herein, the phrase, “media guidance data” or “guidance data” should be understood to mean any data related to content, such as media listings, media-related information (e.g., broadcast times, broadcast channels, titles, descriptions, ratings information (e.g., parental control ratings, critic's ratings, etc.), genre or category information, actor information, logo data for broadcasters' or providers' logos, etc.), media format (e.g., standard definition, high definition, 3D, etc.), advertisement information (e.g., text, images, media clips, etc.), on-demand information, blogs, websites, and any other type of guidance data that is helpful for a user to navigate among and locate desired content selections.

Other operations of a media guidance application are to play media assets and provide fast access playback operations for those media assets. As referred to herein, the phrase “fast-access playback operations” should be understood to mean any operation that pertains to playing back a non-linear media asset faster than normal playback speed or in a different order than the media asset is designed to be played, such as a fast-forward, rewind, skip, chapter selection, segment selection, skip segment, jump segment, next segment, previous segment, skip advertisement or commercial, next chapter, previous chapter or any other operation that does not play back the media asset at normal playback speed. The fast-access playback operation may be any playback operation that is not “play,” where the play operation plays back the media asset at normal playback speed.

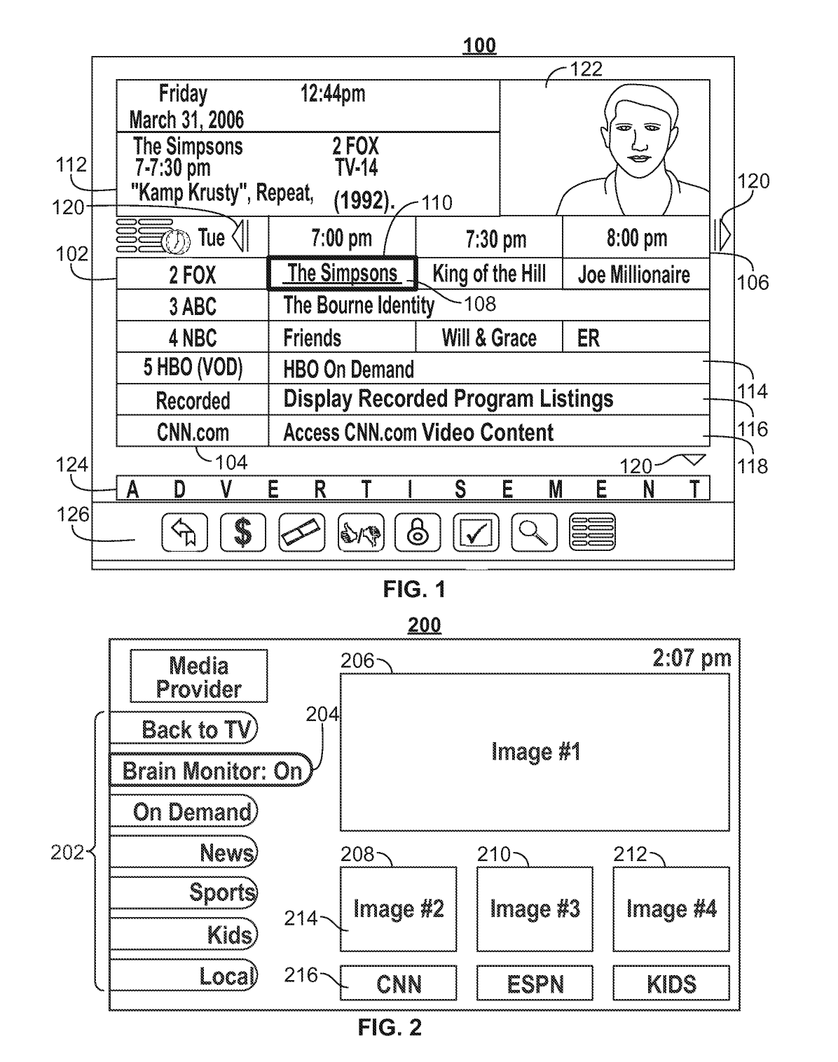

FIGS. 1 and 2 show illustrative display screens that may be used to provide media guidance data. The display screens shown in FIGS. 1 and 2 may be implemented on any suitable user equipment device or platform. While the displays of FIGS. 1 and 2 are illustrated as full screen displays, they may also be fully or partially overlaid over content being displayed. A user may indicate a desire to access content information by selecting a selectable option provided in a display screen (e.g., a menu option, a listings option, an icon, a hyperlink, etc.) or pressing a dedicated button (e.g., a GUIDE button) on a remote control or other user input interface or device. In response to the user's indication, the media guidance application may provide a display screen with media guidance data organized in one of several ways, such as by time and channel in a grid, by time, by channel, by source, by content type, by category (e.g., movies, sports, news, children, or other categories of programming), or other predefined, user-defined, or other organization criteria. The organization of the media guidance data is determined by guidance application data. As referred to herein, the phrase, “guidance application data” should be understood to mean data used in operating the guidance application, such as program information, guidance application settings, user preferences, or user profile information.

FIG. 1 shows illustrative grid program listings display 100 arranged by time and channel that also enables access to different types of content in a single display. Display 100 may include grid 102 with: (1) a column of channel/content type identifiers 104, where each channel/content type identifier (which is a cell in the column) identifies a different channel or content type available; and (2) a row of time identifiers 106, where each time identifier (which is a cell in the row) identifies a time block of programming. Grid 102 also includes cells of program listings, such as program listing 108, where each listing provides the title of the program provided on the listing's associated channel and time. With a user input device, a user can select program listings by moving highlight region 110. Information relating to the program listing selected by highlight region 110 may be provided in program information region 112. Region 112 may include, for example, the program title, the program description, the time the program is provided (if applicable), the channel the program is on (if applicable), the program's rating, and other desired information.

In addition to providing access to linear programming (e.g., content that is scheduled to be transmitted to a plurality of user equipment devices at a predetermined time and is provided according to a schedule), the media guidance application also provides access to non-linear programming (e.g., content accessible to a user equipment device at any time and is not provided according to a schedule). Non-linear programming may include content from different content sources including on-demand content (e.g., VOD), Internet content (e.g., streaming media, downloadable media, etc.), locally stored content (e.g., content stored on any user equipment device described above or other storage device), or other time-independent content. On-demand content may include movies or any other content provided by a particular content provider (e.g., HBO On Demand providing “The Sopranos” and “Curb Your Enthusiasm”). HBO ON DEMAND is a service mark owned by Time Warner Company L.P. et al. and THE SOPRANOS and CURB YOUR ENTHUSIASM are trademarks owned by the Home Box Office, Inc. Internet content may include web events, such as a chat session or Webcast, or content available on-demand as streaming content or downloadable content through an Internet web site or other Internet access (e.g. FTP).

Grid 102 may provide media guidance data for non-linear programming including on-demand listing 114, recorded content listing 116, and Internet content listing 118. A display combining media guidance data for content from different types of content sources is sometimes referred to as a “mixed-media” display. Various permutations of the types of media guidance data that may be displayed that are different than display 100 may be based on user selection or guidance application definition (e.g., a display of only recorded and broadcast listings, only on-demand and broadcast listings, etc.). As illustrated, listings 114, 116, and 118 are shown as spanning the entire time block displayed in grid 102 to indicate that selection of these listings may provide access to a display dedicated to on-demand listings, recorded listings, or Internet listings, respectively. In some embodiments, listings for these content types may be included directly in grid 102. Additional media guidance data may be displayed in response to the user selecting one of the navigational icons 120. (Pressing an arrow key on a user input device may affect the display in a similar manner as selecting navigational icons 120.)

Display 100 may also include video region 122, advertisement 124, and options region 126. Video region 122 may allow the user to view and/or preview programs that are currently available, will be available, or were available to the user. The content of video region 122 may correspond to, or be independent from, one of the listings displayed in grid 102. Grid displays including a video region are sometimes referred to as picture-in-guide (PIG) displays. PIG displays and their functionalities are described in greater detail in Satterfield et al. U.S. Pat. No. 6,564,378, issued May 13, 2003 and Yuen et al. U.S. Pat. No. 6,239,794, issued May 29, 2001, which are hereby incorporated by reference herein in their entireties. PIG displays may be included in other media guidance application display screens of the embodiments described herein.

Advertisement 124 may provide an advertisement for content that, depending on a viewer's access rights (e.g., for subscription programming), is currently available for viewing, will be available for viewing in the future, or may never become available for viewing, and may correspond to or be unrelated to one or more of the content listings in grid 102. Advertisement 124 may also be for products or services related or unrelated to the content displayed in grid 102. Advertisement 124 may be selectable and provide further information about content, provide information about a product or a service, enable purchasing of content, a product, or a service, provide content relating to the advertisement, etc. Advertisement 124 may be targeted based on a user's profile/preferences, monitored user activity, the type of display provided, or on other suitable targeted advertisement bases.

While advertisement 124 is shown as rectangular or banner shaped, advertisements may be provided in any suitable size, shape, and location in a guidance application display. For example, advertisement 124 may be provided as a rectangular shape that is horizontally adjacent to grid 102. This is sometimes referred to as a panel advertisement. In addition, advertisements may be overlaid over content or a guidance application display or embedded within a display. Advertisements may also include text, images, rotating images, video clips, or other types of content described above. Advertisements may be stored in a user equipment device having a guidance application, in a database connected to the user equipment, in a remote location (including streaming media servers), or on other storage means, or a combination of these locations. Providing advertisements in a media guidance application is discussed in greater detail in, for example, Knudson et al., U.S. Patent Application Publication No. 2003/0110499, filed Jan. 17, 2003; Ward, III et al. U.S. Pat. No. 6,756,997, issued Jun. 29, 2004; and Schein et al. U.S. Pat. No. 6,388,714, issued May 14, 2002, which are hereby incorporated by reference herein in their entireties. It will be appreciated that advertisements may be included in other media guidance application display screens of the embodiments described herein.

Options region 126 may allow the user to access different types of content, media guidance application displays, and/or media guidance application features. Options region 126 may be part of display 100 (and other display screens described herein), or may be invoked by a user by selecting an on-screen option or pressing a dedicated or assignable button on a user input device. The selectable options within options region 126 may concern features related to program listings in grid 102 or may include options available from a main menu display. Features related to program listings may include searching for other air times or ways of receiving a program, recording a program, enabling series recording of a program, setting program and/or channel as a favorite, purchasing a program, or other features. Options available from a main menu display may include search options, VOD options, parental control options, Internet options, cloud-based options, device synchronization options, second screen device options, options to access various types of media guidance data displays, options to subscribe to a premium service, options to edit a user's profile, options to access a browse overlay, or other options.

The media guidance application may be personalized based on a user's preferences. A personalized media guidance application allows a user to customize displays and features to create a personalized “experience” with the media guidance application. This personalized experience may be created by allowing a user to input these customizations and/or by the media guidance application monitoring user activity to determine various user preferences. Users may access their personalized guidance application by logging in or otherwise identifying themselves to the guidance application. Customization of the media guidance application may be made in accordance with a user profile. The customizations may include varying presentation schemes (e.g., color scheme of displays, font size of text, etc.), aspects of content listings displayed (e.g., only HDTV or only 3D programming, user-specified broadcast channels based on favorite channel selections, re-ordering the display of channels, recommended content, etc.), desired recording features (e.g., recording or series recordings for particular users, recording quality, etc.), parental control settings, customized presentation of Internet content (e.g., presentation of social media content, e-mail, electronically delivered articles, etc.) and other desired customizations.

The media guidance application may allow a user to provide user profile information or may automatically compile user profile information. The media guidance application may, for example, monitor the content the user accesses and/or other interactions the user may have with the guidance application. Additionally, the media guidance application may obtain all or part of other user profiles that are related to a particular user (e.g., from other web sites on the Internet the user accesses, such as www.allrovi.com, from other media guidance applications the user accesses, from other interactive applications the user accesses, from another user equipment device of the user, etc.), and/or obtain information about the user from other sources that the media guidance application may access. As a result, a user can be provided with a unified guidance application experience across the user's different user equipment devices. This type of user experience is described in greater detail below in connection with FIG. 4. Additional personalized media guidance application features are described in greater detail in Ellis et al., U.S. Patent Application Publication No. 2005/0251827, filed Jul. 11, 2005, Boyer et al., U.S. Pat. No. 7,165,098, issued Jan. 16, 2007, and Ellis et al., U.S. Patent Application Publication No. 2002/0174430, filed Feb. 21, 2002, which are hereby incorporated by reference herein in their entireties.

Another display arrangement for providing media guidance is shown in FIG. 2. Video mosaic display 200 includes selectable options 202 for content information organized based on content type, genre, and/or other organization criteria. In display 200, selectable option 204 is selected, thus initiating brain monitoring of a user.

In display 200 listings may provide graphical images including cover art, still images from the content, video clip previews, live video from the content, or other types of content that indicate to a user the content being described by the media guidance data in the listing. Each of the graphical listings may also be accompanied by text to provide further information about the content associated with the listing. For example, listings 208, 210, and 212 may include more than one portion, including media portion 214 and text portion 216. Media portion 214 and/or text portion 216 may be selectable to view content in full-screen or to view information related to the content displayed in media portion 214 (e.g., to view listings for the channel that the video is displayed on).

The listings in display 200 are of different sizes (i.e., listing 206 is larger than listings 208, 210, and 212), but if desired, all the listings may be the same size. Listings may be of different sizes or graphically accentuated to indicate degrees of interest to the user or to emphasize certain content, as desired by the content provider or based on user preferences. Various systems and methods for graphically accentuating content listings are discussed in, for example, Yates, U.S. Patent Application Publication No. 2010/0153885, filed Dec. 29, 2005, which is hereby incorporated by reference herein in its entirety.

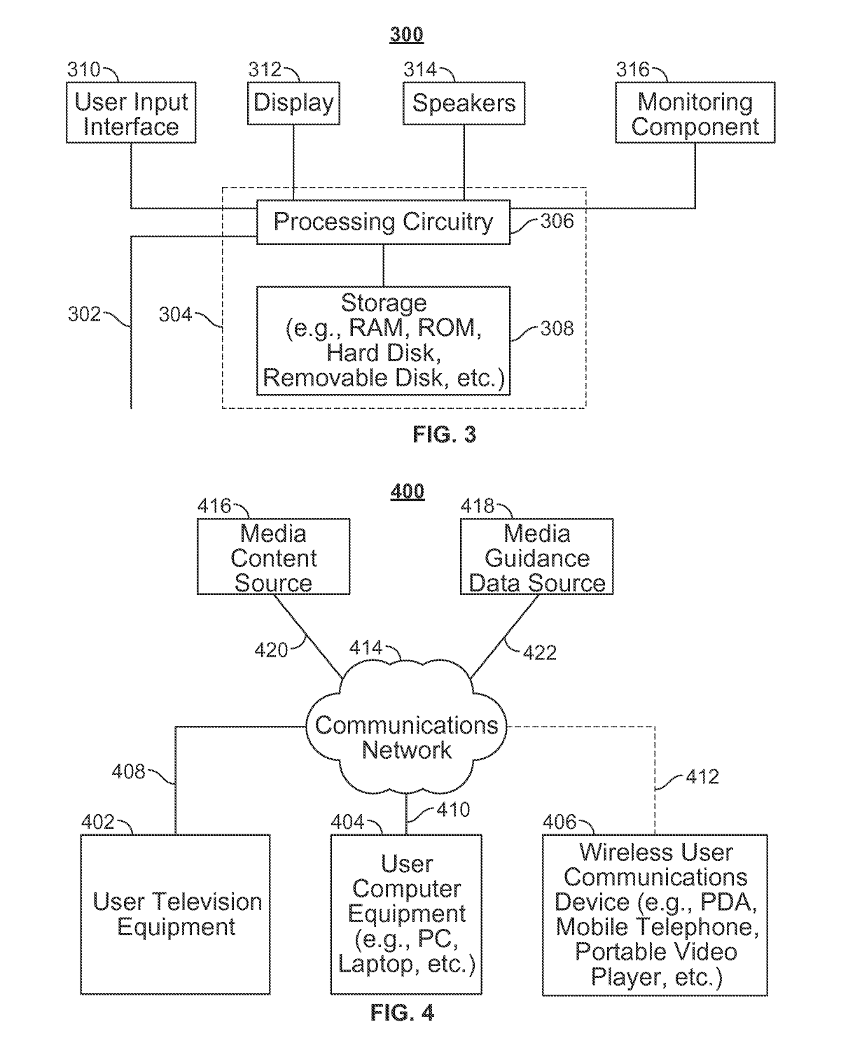

Users may access content and the media guidance application (and its display screens described above and below) from one or more of their user equipment devices. FIG. 3 shows a generalized embodiment of illustrative user equipment device 300. More specific implementations of user equipment devices are discussed below in connection with FIG. 4. User equipment device 300 may receive content and data via input/output (hereinafter “I/O”) path 302. I/O path 302 may provide content (e.g., broadcast programming, on-demand programming, Internet content, content available over a local area network (LAN) or wide area network (WAN), and/or other content) and data to control circuitry 304, which includes processing circuitry 306 and storage 308. Control circuitry 304 may be used to send and receive commands, requests, and other suitable data using I/O path 302. I/O path 302 may connect control circuitry 304 (and specifically processing circuitry 306) to one or more communications paths (described below). I/O functions may be provided by one or more of these communications paths, but are shown as a single path in FIG. 3 to avoid overcomplicating the drawing.

Control circuitry 304 may be based on any suitable processing circuitry such as processing circuitry 306. As referred to herein, processing circuitry should be understood to mean circuitry based on one or more microprocessors, microcontrollers, digital signal processors, programmable logic devices, field-programmable gate arrays (FPGAs), application-specific integrated circuits (ASICs), etc., and may include a multi-core processor (e.g., dual-core, quad-core, hexa-core, or any suitable number of cores) or supercomputer. In some embodiments, processing circuitry may be distributed across multiple separate processors or processing units, for example, multiple of the same type of processing units (e.g., two Intel Core i7 processors) or multiple different processors (e.g., an Intel Core i5 processor and an Intel Core i7 processor). In some embodiments, control circuitry 304 executes instructions for a media guidance application stored in memory (i.e., storage 308). Specifically, control circuitry 304 may be instructed by the media guidance application to perform the functions discussed above and below. For example, the media guidance application may provide instructions to control circuitry 304 to generate the media guidance displays. In some implementations, any action performed by control circuitry 304 may be based on instructions received from the media guidance application.

In client-server based embodiments, control circuitry 304 may include communications circuitry suitable for communicating with a guidance application server or other networks or servers. The instructions for carrying out the above mentioned functionality may be stored on the guidance application server. Communications circuitry may include a cable modem, an integrated services digital network (ISDN) modem, a digital subscriber line (DSL) modem, a telephone modem, Ethernet card, or a wireless modem for communications with other equipment, or any other suitable communications circuitry. Such communications may involve the Internet or any other suitable communications networks or paths (which is described in more detail in connection with FIG. 4). In addition, communications circuitry may include circuitry that enables peer-to-peer communication of user equipment devices, or communication of user equipment devices in locations remote from each other (described in more detail below).

Memory may be an electronic storage device provided as storage 308 that is part of control circuitry 304. As referred to herein, the phrase “electronic storage device” or “storage device” should be understood to mean any device for storing electronic data, computer software, or firmware, such as random-access memory, read-only memory, hard drives, optical drives, digital video disc (DVD) recorders, compact disc (CD) recorders, BLU-RAY disc (BD) recorders, BLU-RAY 3D disc recorders, digital video recorders (DVR, sometimes called a personal video recorder, or PVR), solid state devices, quantum storage devices, gaming consoles, gaming media, or any other suitable fixed or removable storage devices, and/or any combination of the same. Storage 308 may be used to store various types of content described herein as well as media guidance information, described above, and guidance application data, described above. Nonvolatile memory may also be used (e.g., to launch a boot-up routine and other instructions). Cloud-based storage, described in relation to FIG. 4, may be used to supplement storage 308 or instead of storage 308.

Control circuitry 304 may include video generating circuitry and tuning circuitry, such as one or more analog tuners, one or more MPEG-2 decoders or other digital decoding circuitry, high-definition tuners, or any other suitable tuning or video circuits or combinations of such circuits. Encoding circuitry (e.g., for converting over-the-air, analog, or digital signals to MPEG signals for storage) may also be provided. Control circuitry 304 may also include scaler circuitry for upconverting and downconverting content into the preferred output format of the user equipment 300. Circuitry 304 may also include digital-to-analog converter circuitry and analog-to-digital converter circuitry for converting between digital and analog signals. The tuning and encoding circuitry may be used by the user equipment device to receive and to display, to play, or to record content. The tuning and encoding circuitry may also be used to receive guidance data. The circuitry described herein, including for example, the tuning, video generating, encoding, decoding, encrypting, decrypting, scaler, and analog/digital circuitry, may be implemented using software running on one or more general purpose or specialized processors. Multiple tuners may be provided to handle simultaneous tuning functions (e.g., watch and record functions, picture-in-picture (PIP) functions, multiple-tuner recording, etc.). If storage 308 is provided as a separate device from user equipment 300, the tuning and encoding circuitry (including multiple tuners) may be associated with storage 308.

A user may send instructions to control circuitry 304 using user input interface 310. User input interface 310 may be any suitable user interface, such as a remote control, mouse, trackball, keypad, keyboard, touch screen, touchpad, stylus input, joystick, voice recognition interface, or other user input interfaces. Display 312 may be provided as a stand-alone device or integrated with other elements of user equipment device 300. Display 312 may be one or more of a monitor, a television, a liquid crystal display (LCD) for a mobile device, or any other suitable equipment for displaying visual images. In some embodiments, display 312 may be HDTV-capable. In some embodiments, display 312 may be a 3D display, and the interactive media guidance application and any suitable content may be displayed in 3D. A video card or graphics card may generate the output to the display 312. The video card may offer various functions such as accelerated rendering of 3D scenes and 2D graphics, MPEG-2/MPEG-4 decoding, TV output, or the ability to connect multiple monitors. The video card may be any processing circuitry described above in relation to control circuitry 304. The video card may be integrated with the control circuitry 304. Speakers 314 may be provided as integrated with other elements of user equipment device 300 or may be stand-alone units. The audio component of videos and other content displayed on display 312 may be played through speakers 314. In some embodiments, the audio may be distributed to a receiver (not shown), which processes and outputs the audio via speakers 314.

Control circuitry 304 may also instruct monitoring component 316. Monitoring component 316 may include one or more additional sub-components (e.g., an EEG, EMG, etc.) for monitoring brain activity of a user. Monitoring component 316 may transmit updates (e.g., associated with brain activity) of a user to control circuitry 304. Control circuitry 304 may compare the updates to data related to brain activity (e.g., threshold ranges, frequency ranges, etc.) of the user and/or other users stored on storage 308 (e.g., to determine whether or not the brain activity of the user corresponds to a particular threshold range and/or mood, attentiveness level, etc.).

It should be noted, monitoring component 316 may, in some embodiments, be located on a separate device in communication with the device upon which a media guidance application (and control circuitry 304) is implemented. For example, in some embodiments, monitoring component 316 may communicate with device 300 via a communications network (e.g., communications network 414 (FIG. 4)).

The guidance application may be implemented using any suitable architecture. For example, it may be a stand-alone application wholly implemented on user equipment device 300. In such an approach, instructions of the application are stored locally, and data for use by the application is downloaded on a periodic basis (e.g., from an out-of-band feed, from an Internet resource, or using another suitable approach). In some embodiments, the media guidance application is a client-server based application. Data for use by a thick or thin client implemented on user equipment device 300 is retrieved on-demand by issuing requests to a server remote to the user equipment device 300. In one example of a client-server based guidance application, control circuitry 304 runs a web browser that interprets web pages provided by a remote server.

In some embodiments, the media guidance application is downloaded and interpreted or otherwise run by an interpreter or virtual machine (run by control circuitry 304). In some embodiments, the guidance application may be encoded in the ETV Binary Interchange Format (EBIF), received by control circuitry 304 as part of a suitable feed, and interpreted by a user agent running on control circuitry 304. For example, the guidance application may be an EBIF application. In some embodiments, the guidance application may be defined by a series of JAVA-based files that are received and run by a local virtual machine or other suitable middleware executed by control circuitry 304. In some of such embodiments (e.g., those employing MPEG-2 or other digital media encoding schemes), the guidance application may be, for example, encoded and transmitted in an MPEG-2 object carousel with the MPEG audio and video packets of a program.

User equipment device 300 of FIG. 3 can be implemented in system 400 of FIG. 4 as user television equipment 402, user computer equipment 404, wireless user communications device 406, or any other type of user equipment suitable for accessing content, such as a non-portable gaming machine. For simplicity, these devices may be referred to herein collectively as user equipment or user equipment devices, and may be substantially similar to user equipment devices described above. User equipment devices, on which a media guidance application may be implemented, may function as a standalone device or may be part of a network of devices. Various network configurations of devices may be implemented and are discussed in more detail below.

A user equipment device utilizing at least some of the system features described above in connection with FIG. 3 may not be classified solely as user television equipment 402, user computer equipment 404, or a wireless user communications device 406. For example, user television equipment 402 may, like some user computer equipment 404, be Internet-enabled allowing for access to Internet content, while user computer equipment 404 may, like some television equipment 402, include a tuner allowing for access to television programming. The media guidance application may have the same layout on various different types of user equipment or may be tailored to the display capabilities of the user equipment. For example, on user computer equipment 404, the guidance application may be provided as a web site accessed by a web browser. In another example, the guidance application may be scaled down for wireless user communications devices 406.

In system 400, there is typically more than one of each type of user equipment device but only one of each is shown in FIG. 4 to avoid overcomplicating the drawing. In addition, each user may utilize more than one type of user equipment device and also more than one of each type of user equipment device.

In some embodiments, a user equipment device (e.g., user television equipment 402, user computer equipment 404, wireless user communications device 406) may be referred to as a “second screen device.” For example, a second screen device may supplement content presented on a first user equipment device. The content presented on the second screen device may be any suitable content that supplements the content presented on the first device. In some embodiments, the second screen device provides an interface for adjusting settings and display preferences of the first device. In some embodiments, the second screen device is configured for interacting with other second screen devices or for interacting with a social network. The second screen device can be located in the same room as the first device, a different room from the first device but in the same house or building, or in a different building from the first device.

The user may also set various settings to maintain consistent media guidance application settings across in-home devices and remote devices. Settings include those described herein, as well as channel and program favorites, programming preferences that the guidance application utilizes to make programming recommendations, display preferences, and other desirable guidance settings. For example, if a user sets a channel as a favorite on, for example, the web site www.allrovi.com on their personal computer at their office, the same channel would appear as a favorite on the user's in-home devices (e.g., user television equipment and user computer equipment) as well as the user's mobile devices, if desired. Therefore, changes made on one user equipment device can change the guidance experience on another user equipment device, regardless of whether they are the same or a different type of user equipment device. In addition, the changes made may be based on settings input by a user, as well as user activity monitored by the guidance application.

The user equipment devices may be coupled to communications network 414. Namely, user television equipment 402, user computer equipment 404, and wireless user communications device 406 are coupled to communications network 414 via communications paths 408, 410, and 412, respectively. Communications network 414 may be one or more networks including the Internet, a mobile phone network, mobile voice or data network (e.g., a 4G or LTE network), cable network, public switched telephone network, or other types of communications network or combinations of communications networks. Paths 408, 410, and 412 may separately or together include one or more communications paths, such as, a satellite path, a fiber-optic path, a cable path, a path that supports Internet communications (e.g., IPTV), free-space connections (e.g., for broadcast or other wireless signals), or any other suitable wired or wireless communications path or combination of such paths. Path 412 is drawn with dotted lines to indicate that in the exemplary embodiment shown in FIG. 4 it is a wireless path and paths 408 and 410 are drawn as solid lines to indicate they are wired paths (although these paths may be wireless paths, if desired). Communications with the user equipment devices may be provided by one or more of these communications paths, but are shown as a single path in FIG. 4 to avoid overcomplicating the drawing.

Although communications paths are not drawn between user equipment devices, these devices may communicate directly with each other via communication paths, such as those described above in connection with paths 408, 410, and 412, as well as other short-range point-to-point communication paths, such as USB cables, IEEE 1394 cables, wireless paths (e.g., Bluetooth, infrared, IEEE 802-11x, etc.), or other short-range communication via wired or wireless paths. BLUETOOTH is a certification mark owned by Bluetooth SIG, INC. The user equipment devices may also communicate with each other directly through an indirect path via communications network 414.

System 400 includes content source 416 and media guidance data source 418 coupled to communications network 414 via communication paths 420 and 422, respectively. Paths 420 and 422 may include any of the communication paths described above in connection with paths 408, 410, and 412. Communications with the content source 416 and media guidance data source 418 may be exchanged over one or more communications paths, but are shown as a single path in FIG. 4 to avoid overcomplicating the drawing. In addition, there may be more than one of each of content source 416 and media guidance data source 418, but only one of each is shown in FIG. 4 to avoid overcomplicating the drawing. (The different types of each of these sources are discussed below.) If desired, content source 416 and media guidance data source 418 may be integrated as one source device. Although communications between sources 416 and 418 with user equipment devices 402, 404, and 406 are shown as through communications network 414, in some embodiments, sources 416 and 418 may communicate directly with user equipment devices 402, 404, and 406 via communication paths (not shown) such as those described above in connection with paths 408, 410, and 412.

Content source 416 may include one or more types of content distribution equipment including a television distribution facility, cable system headend, satellite distribution facility, programming sources (e.g., television broadcasters, such as NBC, ABC, HBO, etc.), intermediate distribution facilities and/or servers, Internet providers, on-demand media servers, and other content providers. NBC is a trademark owned by the National Broadcasting Company, Inc., ABC is a trademark owned by the American Broadcasting Company, Inc., and HBO is a trademark owned by the Home Box Office, Inc. Content source 416 may be the originator of content (e.g., a television broadcaster, a Webcast provider, etc.) or may not be the originator of content (e.g., an on-demand content provider, an Internet provider of content of broadcast programs for downloading, etc.). Content source 416 may include cable sources, satellite providers, on-demand providers, Internet providers, over-the-top content providers, or other providers of content. Content source 416 may also include a remote media server used to store different types of content (including video content selected by a user), in a location remote from any of the user equipment devices. Systems and methods for remote storage of content, and providing remotely stored content to user equipment are discussed in greater detail in connection with Ellis et al., U.S. Pat. No. 7,761,892, issued Jul. 20, 2010, which is hereby incorporated by reference herein in its entirety.

Media guidance data source 418 may provide media guidance data, such as the media guidance data described above. Media guidance application data may be provided to the user equipment devices using any suitable approach. In some embodiments, the guidance application may be a stand-alone interactive television program guide that receives program guide data via a data feed (e.g., a continuous feed or trickle feed). Program schedule data and other guidance data may be provided to the user equipment on a television channel sideband, using an in-band digital signal, using an out-of-band digital signal, or by any other suitable data transmission technique. Program schedule data and other media guidance data may be provided to user equipment on multiple analog or digital television channels.

In some embodiments, guidance data from media guidance data source 418 may be provided to users' equipment using a client-server approach. For example, a user equipment device may pull media guidance data from a server, or a server may push media guidance data to a user equipment device. In some embodiments, a guidance application client residing on the user's equipment may initiate sessions with source 418 to obtain guidance data when needed, e.g., when the guidance data is out of date or when the user equipment device receives a request from the user to receive data. Media guidance may be provided to the user equipment with any suitable frequency (e.g., continuously, daily, a user-specified period of time, a system-specified period of time, in response to a request from user equipment, etc.). Media guidance data source 418 may provide user equipment devices 402, 404, and 406 the media guidance application itself or software updates for the media guidance application.

Media guidance applications may be, for example, stand-alone applications implemented on user equipment devices. For example, the media guidance application may be implemented as software or a set of executable instructions which may be stored in storage 308, and executed by control circuitry 304 of a user equipment device 300. In some embodiments, media guidance applications may be client-server applications where only a client application resides on the user equipment device, and server application resides on a remote server. For example, media guidance applications may be implemented partially as a client application on control circuitry 304 of user equipment device 300 and partially on a remote server as a server application (e.g., media guidance data source 418) running on control circuitry of the remote server. When executed by control circuitry of the remote server (such as media guidance data source 418), the media guidance application may instruct the control circuitry to generate the guidance application displays and transmit the generated displays to the user equipment devices. The server application may instruct the control circuitry of the media guidance data source 418 to transmit data for storage on the user equipment. The client application may instruct control circuitry of the receiving user equipment to generate the guidance application displays.

Content and/or media guidance data delivered to user equipment devices 402, 404, and 406 may be over-the-top (OTT) content. OTT content delivery allows Internet-enabled user devices, including any user equipment device described above, to receive content that is transferred over the Internet, including any content described above, in addition to content received over cable or satellite connections. OTT content is delivered via an Internet connection provided by an Internet service provider (ISP), but a third party distributes the content. The ISP may not be responsible for the viewing abilities, copyrights, or redistribution of the content, and may only transfer IP packets provided by the OTT content provider. Examples of OTT content providers include YOUTUBE, NETFLIX, and HULU, which provide audio and video via IP packets. Youtube is a trademark owned by Google Inc., Netflix is a trademark owned by Netflix Inc., and Hulu is a trademark owned by Hulu, LLC. OTT content providers may additionally or alternatively provide media guidance data described above. In addition to content and/or media guidance data, providers of OTT content can distribute media guidance applications (e.g., web-based applications or cloud-based applications), or the content can be displayed by media guidance applications stored on the user equipment device.

Media guidance system 400 is intended to illustrate a number of approaches, or network configurations, by which user equipment devices and sources of content and guidance data may communicate with each other for the purpose of accessing content and providing media guidance. The embodiments described herein may be applied in any one or a subset of these approaches, or in a system employing other approaches for delivering content and providing media guidance. The following four approaches provide specific illustrations of the generalized example of FIG. 4.

In one approach, user equipment devices may communicate with each other within a home network. User equipment devices can communicate with each other directly via short-range point-to-point communication schemes described above, via indirect paths through a hub or other similar device provided on a home network, or via communications network 414. Each of the multiple individuals in a single home may operate different user equipment devices on the home network. As a result, it may be desirable for various media guidance information or settings to be communicated between the different user equipment devices. For example, it may be desirable for users to maintain consistent media guidance application settings on different user equipment devices within a home network, as described in greater detail in Ellis et al., U.S. patent application Ser. No. 11/179,410, filed Jul. 11, 2005. Different types of user equipment devices in a home network may also communicate with each other to transmit content. For example, a user may transmit content from user computer equipment to a portable video player or portable music player.

In a second approach, users may have multiple types of user equipment by which they access content and obtain media guidance. For example, some users may have home networks that are accessed by in-home and mobile devices. Users may control in-home devices via a media guidance application implemented on a remote device. For example, users may access an online media guidance application on a website via a personal computer at their office, or a mobile device such as a PDA or web-enabled mobile telephone. The user may set various settings (e.g., recordings, reminders, or other settings) on the online guidance application to control the user's in-home equipment. The online guide may control the user's equipment directly, or by communicating with a media guidance application on the user's in-home equipment. Various systems and methods for user equipment devices communicating, where the user equipment devices are in locations remote from each other, is discussed in, for example, Ellis et al., U.S. Pat. No. 8,046,801, issued Oct. 25, 2011, which is hereby incorporated by reference herein in its entirety.

In a third approach, users of user equipment devices inside and outside a home can use their media guidance application to communicate directly with content source 416 to access content. Specifically, within a home, users of user television equipment 402 and user computer equipment 404 may access the media guidance application to navigate among and locate desirable content. Users may also access the media guidance application outside of the home using wireless user communications devices 406 to navigate among and locate desirable content.

In a fourth approach, user equipment devices may operate in a cloud computing environment to access cloud services. In a cloud computing environment, various types of computing services for content sharing, storage or distribution (e.g., video sharing sites or social networking sites) are provided by a collection of network-accessible computing and storage resources, referred to as “the cloud.” For example, the cloud can include a collection of server computing devices, which may be located centrally or at distributed locations that provide cloud-based services to various types of users and devices connected via a network such as the Internet via communications network 414. These cloud resources may include one or more content sources 416 and one or more media guidance data sources 418. In addition or in the alternative, the remote computing sites may include other user equipment devices, such as user television equipment 402, user computer equipment 404, and wireless user communications device 406. For example, the other user equipment devices may provide access to a stored copy of a video or a streamed video. In such embodiments, user equipment devices may operate in a peer-to-peer manner without communicating with a central server.

The cloud provides access to services, such as content storage, content sharing, or social networking services, among other examples, as well as access to any content described above, for user equipment devices. Services can be provided in the cloud through cloud computing service providers, or through other providers of online services. For example, the cloud-based services can include a content storage service, a content sharing site, a social networking site, or other services via which user-sourced content is distributed for viewing by others on connected devices. These cloud-based services may allow a user equipment device to store content to the cloud and to receive content from the cloud rather than storing content locally and accessing locally-stored content.

A user may use various content capture devices, such as camcorders, digital cameras with video mode, audio recorders, mobile phones, and handheld computing devices, to record content. The user can upload content to a content storage service on the cloud either directly, for example, from user computer equipment 404 or wireless user communications device 406 having content capture feature. Alternatively, the user can first transfer the content to a user equipment device, such as user computer equipment 404. The user equipment device storing the content uploads the content to the cloud using a data transmission service on communications network 414. In some embodiments, the user equipment device itself is a cloud resource, and other user equipment devices can access the content directly from the user equipment device on which the user stored the content.

Cloud resources may be accessed by a user equipment device using, for example, a web browser, a media guidance application, a desktop application, a mobile application, and/or any combination of access applications of the same. The user equipment device may be a cloud client that relies on cloud computing for application delivery, or the user equipment device may have some functionality without access to cloud resources. For example, some applications running on the user equipment device may be cloud applications, i.e., applications delivered as a service over the Internet, while other applications may be stored and run on the user equipment device. In some embodiments, a user device may receive content from multiple cloud resources simultaneously. For example, a user device can stream audio from one cloud resource while downloading content from a second cloud resource. Or a user device can download content from multiple cloud resources for more efficient downloading. In some embodiments, user equipment devices can use cloud resources for processing operations such as the processing operations performed by processing circuitry described in relation to FIG. 3.

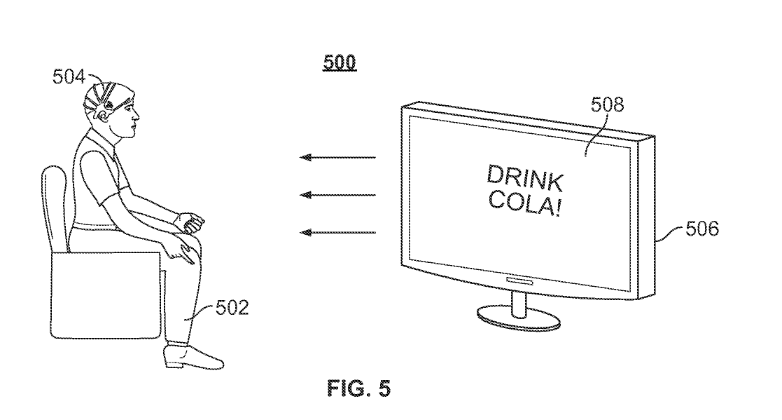

FIG. 5 shows an illustrative representation 500 of monitoring brain activity of a viewer during an advertisement to determine whether the viewer watched the advertisement. Viewer 502 is wearing user device 504 associated with monitoring brain activity (e.g., monitoring component 316 (FIG. 3)). When advertisement 508 including a stimulus is displayed on viewer's television 506, a region of the viewer's brain may be triggered. User device 504 may detect the triggering of the region of the viewer's brain in response to the stimulus. User device 504 may compare brain activity for the region of the viewer's brain to a threshold range associated with the stimulus to determine whether viewer 502 watched advertisement 508.

User device 504 monitors the brain activity of viewer 502 and determines a brain state of the viewer based on the brain activity. The brain state (e.g., the current frequency range of voltage fluctuations in the brain and/or electrical activity of muscles near the brain at rest and during contraction) may correspond to a region of the viewer's brain during presentation of advertisement 508. User device 504 may select a threshold range (e.g., a target frequency range of voltage fluctuations in the brain and/or electrical activity of muscles near the brain at rest and during contraction) that corresponds to the stimulus included in advertisement 508. User device 504 may compare the brain state to the threshold range to determine whether there is a match (e.g., indicative of whether or not viewer 502 viewed advertisement 508).

To determine the advertisement corresponding to a brain state, user device 504 may cross-reference data associated with the brain state of viewer 502 (e.g., the frequency range and/or electrical activity of the muscles near the brain) with a database associated with data related to brain states and corresponding advertisements (e.g., media content source 416 or media guidance data source 418 (FIG. 4)). For example, based on the cross-reference, user device 504 may determine that the data associated with the brain state of the viewer indicates that the viewer watched a COKE advertisement, a FORD advertisement, or another suitable advertisement. User device 504 may then select a threshold range corresponding to presented advertisement 508 based on a time when the advertisement was displayed, a user input indicating the advertisement, metadata associated with the advertisement, or other suitable information. For example, a COKE advertisement may correspond to a particular threshold range while a FORD advertisement may correspond to a different threshold range.

For example, user device 504 monitoring the viewer's brain during advertisement 508 may detect a state of the brain activity associated with the parietal lobe of viewer 502. Advertisement 508 may include a textual stimulus that triggered the parietal lobe of viewer 502 since the parietal lobe is associated with the viewer's reading ability. User device 504 may compare the current brain state of viewer 502 to a threshold range associated with the stimulus included in advertisement 508 to determine whether viewer 502 watched advertisement 508. User device 504 monitoring the viewer's brain activity may receive the threshold range from a database (e.g., media content source 416 or media guidance data source 418 (FIG. 4)). In response to determining that the brain state of viewer 502 matches the threshold range for the stimulus included in advertisement 508, user device 504 may return a message indicating a match and that viewer 502 watched advertisement 508. In response to determining that the brain state of viewer 502 does not match the threshold range for the stimulus included in advertisement 508, user device 504 may return a message indicating no match and that viewer 502 did not watch advertisement 508.

In another example, the stimulus includes a strobe of light, the region includes an occipital lobe of viewer 502, and the threshold range is associated with a response from the occipital lobe in response to the strobe of light displayed during advertisement 508. In this example, the content provider, e.g., the advertiser, includes a strobe of light in advertisement 508. The strobe of light triggers the occipital lobe of the viewer's brain because the occipital lobe is associated with the viewer's vision. User device 504 monitoring the viewer's brain activity receives a threshold range from a database (e.g., media content source 416 or media guidance data source 418 (FIG. 4)) for comparing against the viewer's brain state based on the brain activity. The threshold range is associated with a response from the occipital lobe in response to the strobe of light displayed during advertisement 508. In response to determining that the brain state of viewer 502 matches the threshold range for the stimulus included in advertisement 508, user device 504 may return a message indicating a match and that viewer 502 watched advertisement 508. In response to determining that the brain state of viewer 502 does not match the threshold range for the stimulus included in advertisement 508, user device 504 may return a message indicating no match and that viewer 502 did not watch advertisement 508.

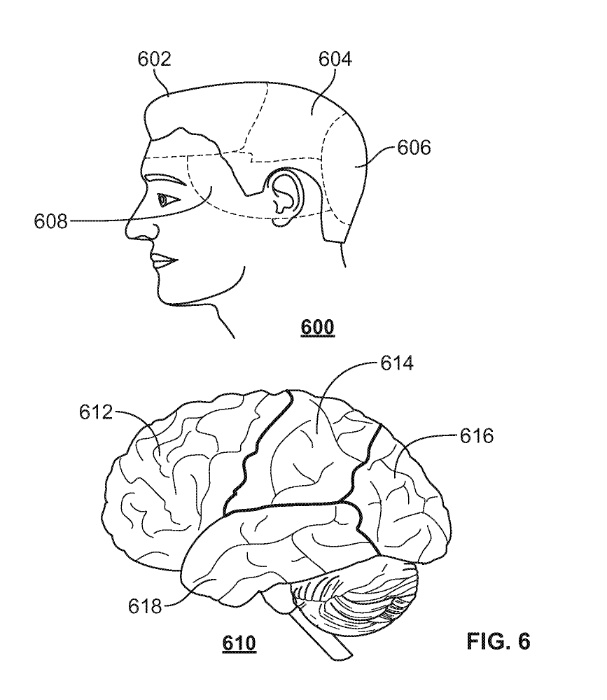

FIG. 6 shows a representation of a user and regions of the brain of the user associated with monitoring brain activity. For example, in some embodiments, a media guidance application may be implemented upon (or be in communication with) a user device that monitors brain activity of a user (e.g., via monitoring component 316 (FIG. 3)). The user device may reside upon the head of a user and include components (or sub-components) for testing different areas of the scalp of a user.

For example, the scalp of user 600 includes first portion 602, second portion 604, third portion 606, and fourth portion 608. In some embodiments, each of first portion 602, second portion 604, third portion 606, and fourth portion 608 may correspond to a different region of brain 610. For example, in some embodiments, first portion 602 may correspond to frontal lobe 612, second portion 604 may correspond to parietal lobe 614, third portion 606 may correspond to occipital lobe 616, and fourth portion 608 may correspond to temporal lobe 618.

In some embodiments, the content provider, e.g., the advertiser, may include different stimuli in an advertisement (e.g., advertisement 508 (FIG. 5)) to trigger the different regions of the user's brain. For example, the content provider may include a visual stimulus in the advertisement to trigger occipital lobe 616 of the user's brain. This is because occipital lobe 616 is associated with the user's vision. In another example, the content provider may include an audio stimulus in the advertisement to trigger temporal lobe 618 of the user's brain. This is because temporal lobe 618 is associated with the user's hearing. In yet another example, the content provider may include a textual stimulus in the advertisement to trigger parietal lobe 614 of the user's brain. This is because parietal lobe 614 is associated with the user's reading ability. In yet another example, the content provider may include an emotional or mood-related stimulus in the advertisement to trigger frontal lobe 612 of the user's brain. This is because frontal lobe 612 is associated with the user's emotions or mood.

In some embodiments, the stimulus in the advertisement (e.g., advertisement 508 (FIG. 5)) includes one or more of a visual stimulus, an audio stimulus, an audiovisual stimulus, a video stimulus, a graphical stimulus, a textual stimulus, or another suitable stimulus. For example, the content provider, e.g., the advertiser, may include a graphical stimulus, such as flashing lights, to trigger the occipital lobe of the viewer. In another example, the content provider, e.g., the advertiser, may include a textual stimulus to trigger the parietal lobe of the viewer. In yet another example, the content provider, e.g., the advertiser, may include an audio stimulus to trigger the temporal lobe of the viewer. The content provider may include any combination of one or more stimuli to trigger various regions of the viewer's brain. The regions triggered by the combination of stimuli may indicate that the viewer watched a particular advertisement. For example, a visual trigger of flashing lights and an audio trigger of a tonal sound may be uniquely associated with a particular advertisement. Detection of corresponding triggers in the occipital and temporal lobes of the viewer may indicate the viewer watched the particular advertisement.

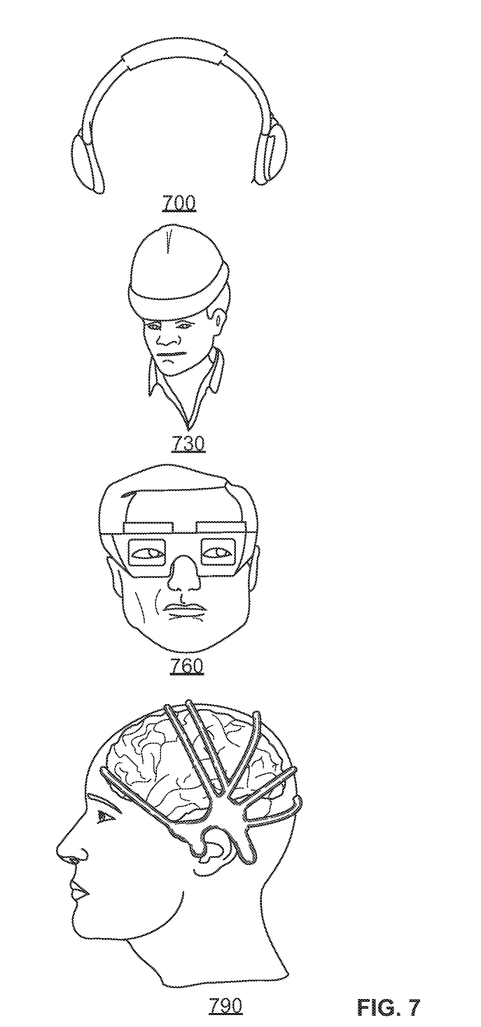

FIG. 7 shows multiple user devices that may be associated with monitoring brain activity. For example, a user device (e.g., upon which a media guidance application is implemented and/or which a media guidance application is in communication with) may be fashioned as a form of headwear.

For example, user devices 700 and 790 are fashioned as headsets, user device 730 is fashioned as a hat/helmet, and user device 760 is fashioned as eye glasses. It should be noted that a user device configured to monitor brain activity as described herein may be fashioned as any headwear. Furthermore, in some embodiments, a user device may not be fashioned as headwear, but instead may be configured as any device capable of monitoring brain activity of a user. For example, any device which may incorporate and/or have access to an EEG, EMG, and/or other means for monitoring brain activity described herein may constitute a user device.

In some embodiments, user devices 700, 730, 760, and 790 may further include additional sub-components (e.g., sub-components of monitoring component 316 (FIG. 3)), which may monitor brain activity on one or more regions of the brain. Sub-components may include electrodes or other features that may attach to the various portions (e.g., portions 602, 604, 606, and 608 (FIG. 6)) of a user (e.g., user 600 (FIG. 6)). Furthermore, in some embodiments, sub-components may extend and/or retract during various modes of the user device in order to accommodate the comfort of the user.

In some embodiments, user devices 700, 730, 760, and 790 may be battery-powered in order to provide a user with additional mobility. Furthermore, user devices 700, 730, 760, and 790 include multiple modes, each corresponding to different power consumption levels and/or sensitivity levels.

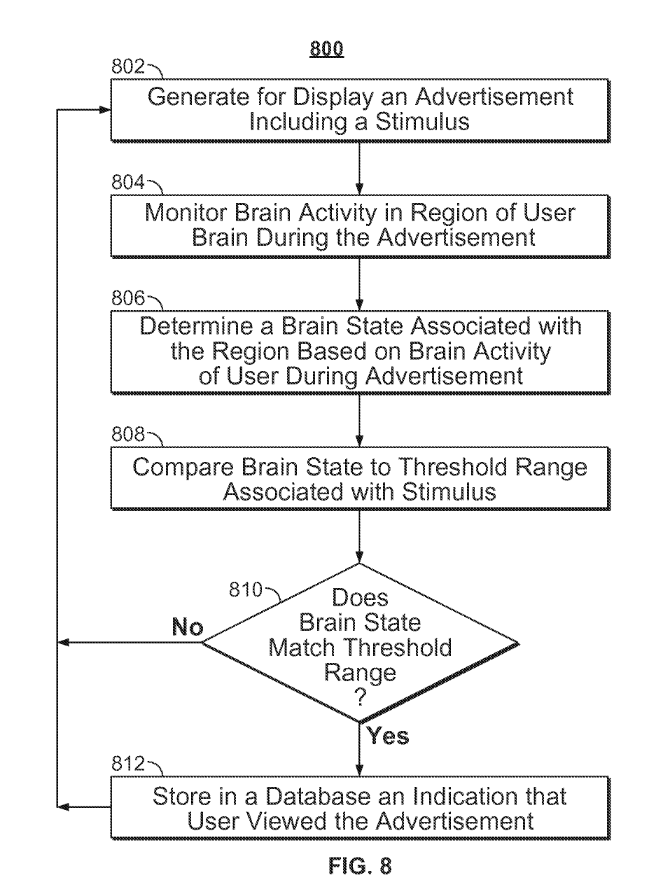

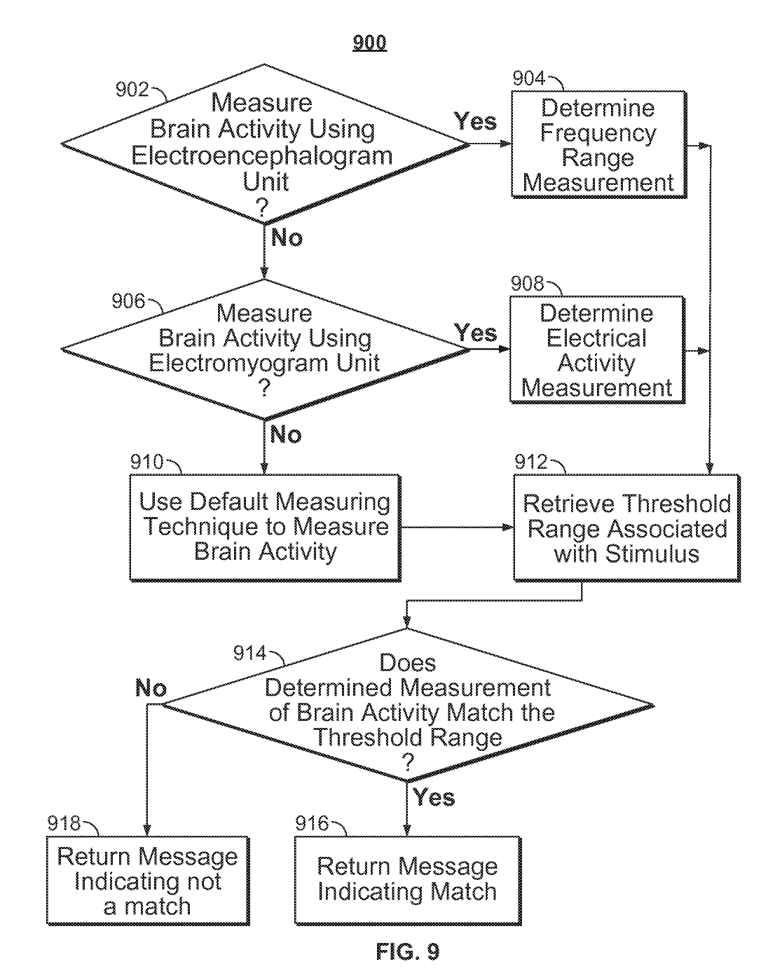

FIG. 8 is a flow-chart of illustrative steps involved in monitoring brain activity of a viewer during an advertisement to determine whether the viewer watched the advertisement. It should be noted that process 800 or any step thereof, could be displayed on, or provided by, any of the devices shown in FIGS. 3-4 and 7 in response to brain activity of a viewer (e.g., user 600 (FIG. 6)). For example, process 800 may be executed by control circuitry 304 (FIG. 3) on user equipment device 402, 404, and/or 406 (FIG. 4) any of which may be configured as headwear (e.g., as shown in FIG. 7) as instructed by the media guidance application while a viewer is consuming media content (e.g., displayed on display 100 (FIG. 1) and/or display 200 (FIG. 2)). In addition, one or more steps of process 800 may be incorporated into or combined with one or more steps of any other process (e.g., as described in FIG. 9).

At step 802, control circuitry 304 (FIG. 3) generates for display an advertisement including a stimulus for triggering a region of a viewer's brain. The stimulus is incorporated into the advertisement by a content provider, e.g., an advertiser, in order to trigger a region of the viewer's brain. The stimulus may include one or more of a visual stimulus, an audio stimulus, an audiovisual stimulus, a video stimulus, a graphical stimulus, a textual stimulus, or another suitable stimulus. For example, the content provider, e.g., the advertiser, may include a graphical stimulus, such as flashing lights, to trigger the occipital lobe of the viewer since it is associated with the viewer's vision.

At step 804, control circuitry 304 monitors brain activity in the region of the viewer's brain during the advertisement. For example, control circuitry 304 may receive data (e.g., from monitoring component 316 (FIG. 3)) associated with the brain activity (e.g., the current frequency range of voltage fluctuations in the brain and/or electrical activity of muscles near the brain at rest and during contraction) of the viewer (e.g., user 600 (FIG. 6)). In some embodiments, monitoring the brain activity of the viewer includes receiving data from an electroencephalogram unit indicating a frequency range of the brain activity of the viewer or receiving data from an electromyogram unit indicating electrical activity of muscles near the brain of the viewer at rest and during contraction. In some embodiments, the electroencephalogram data and the electromyogram data are received from separate devices. In some embodiments, the electroencephalogram data and the electromyogram data are received from the same device.

In some embodiments, control circuitry 304 may continuously monitor the brain activity of a viewer using an EEG, EMG, or suitable device for monitoring brain waves (e.g., incorporated as a sub-component of monitoring component 316 (FIG. 3)). Alternatively, control circuitry 304 may periodically poll the brain activity of a viewer (e.g., on a predetermined schedule and/or in response to a user input, (i.e., selecting selectable option 204 (FIG. 2)).

At step 806, control circuitry 304 determines a brain state associated with the region of the viewer's brain based on the brain activity during the advertisement. For example, control circuitry 304 may receive data from a monitoring component (e.g., monitoring component 316 (FIG. 3)) incorporated into and/or in communication with (e.g., via communications network 414 (FIG. 4) a user device (e.g., user device 300 (FIG. 3) and/or user equipment device 402, 404, and/or 406 (FIG. 4)). Control circuitry 304 may process that data to determine a brain state that corresponds with the received data. For example, the received data may correspond to a particular frequency range and/or electrical activity of the muscles near a particular region (e.g., frontal lobe 612 (FIG. 6)) of the brain (e.g., brain 610 (FIG. 6)) of the viewer.

At step 808, control circuitry 304 compares the determined brain state to a threshold range retrieved from a database stored locally on storage 308 (FIG. 3) or stored remotely at media guidance data source 418 (FIG. 4), and/or any location accessible via communications network 414 (FIG. 4)) associated with frequencies of brain states and corresponding advertisements. Depending on the type of monitoring used, the threshold range for the stimulus may be associated with the frequency range of the brain activity or the electrical activity of muscles near the brain. The threshold range may be stored in a database and, when required, retrieved by a user device monitoring the brain activity of the viewer.

Control circuitry 304 may retrieve quantitative measurements associated with the brain state. For example, in some embodiments, the brain state is associated with a particular frequency range, electrical activity, and/or threshold range. Control circuitry 304 may compare these quantitative measurements to the threshold range to determine whether or not the two measurements match (e.g., within a particular degree of deviation). For example, at step 810, control circuitry 304 may determine whether the two measurements share the same frequency bands. Process 900 (FIG. 9) provides a further illustrative embodiment for steps 808 and 810.

Control circuitry 304 proceeds to step 802 in response to determining that the brain state does not correspond to the threshold range associated with the stimulus. In response to determining that the brain state corresponds to the threshold range associated with the stimulus, at step 812, control circuitry 304 stores in the database an indication that the viewer watched the advertisement. In some embodiments, control circuitry 304 updates a count for the number of instances the viewer has watched an advertisement. For example, after determining which advertisement the viewer has watched, control circuitry 304 may send an indication to the database to update the number of times the viewer has watched the advertisement. The indication may include additional information regarding the advertisement, such as presentation time, presentation channel, and other suitable information. The content provider may utilize the information from the database to improve their future advertising efforts.

In some embodiments, control circuitry 304 cross-references with a database to determine the advertisement the viewer has watched. After control circuitry 304 detects a trigger in a region of the viewer's brain, control circuitry 304 may determine which advertisement the viewer watched. In one example, the database includes reference brain states associated with multiple advertisements. Control circuitry 304 determines which advertisement the viewer has watched by determining which reference brain state matches the viewer's brain state. For example, a reference brain state invoked by a visual trigger of flashing lights and an audio trigger of a tonal sound may be uniquely associated with a particular advertisement. Detection of corresponding triggers in the occipital and temporal lobes of the viewer may indicate the viewer watched the particular advertisement. In another example, the database includes reference metadata associated with multiple advertisements. Control circuitry 304 determines which advertisement the viewer has watched by determining which reference metadata matches the metadata for the advertisement presented to the viewer.

In some embodiments, the database may be stored locally on storage 308 (FIG. 3) or stored remotely at media guidance data source 418 (FIG. 4) and/or any location accessible via communications network 414 (FIG. 4). Additionally or alternatively, the information in the database may be generated by a third party. For example, control circuitry 304 may receive data associated with an advertisement from a remote source (e.g., media content source 416 (FIG. 4)) that indicates a threshold range for a stimulus included in the advertisement.

It is contemplated that the steps or descriptions of FIG. 8 may be used with any other embodiment of this disclosure. In addition, the steps and descriptions described in relation to FIG. 8 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 and 7 could be used to perform one or more of the steps in FIG. 8.

FIG. 9 is a flow-chart of illustrative steps involved in comparing a viewer's brain state to a threshold range associated with a stimulus included in an advertisement. For example, process 900 may correspond to steps 808 and 810 in FIG. 8. It should be noted that process 900 or any step thereof, could be provided by, any of the devices shown in FIGS. 3-4 and 7 in response to brain activity of a user (e.g., user 600 (FIG. 6)). For example, process 900 may be executed by control circuitry 304 (FIG. 3) on user equipment device 402, 404, and/or 406 (FIG. 4) any of which may be configured as headwear (e.g., as shown in FIG. 7) as instructed by the media guidance application while a user is consuming media content (e.g., displayed on display 100 (FIG. 1), and/or display 200 (FIG. 2)). In addition, one or more steps of process 900 may be incorporated into or combined with one or more steps of any other process (e.g. as described in FIG. 8).

At step 902, control circuitry 304 determines whether or not to monitor the brain activity of a user using an EEG. For example, the user device (e.g., user device 700, 730, 760, and/or 790 (FIG. 7)) may include one or more monitoring components (e.g., monitoring component 316 (FIG. 3)) or monitoring sub-components, which may include an EEG. If control circuitry 304 determines to monitor the brain activity of a user using an EEG, control circuitry 304 proceeds to step 904, determines a measurement of a frequency range of the brain activity of a user of one or more regions of the brain of a user, and then proceeds to step 912. If control circuitry 304 determines not to use an EEG (e.g., an EEG is not included in the monitoring component), control circuitry 304 proceeds to step 906.

At step 906, control circuitry 304 determines whether or not to monitor the brain activity of a user using an EMG. For example, the user device (e.g., user device 700, 730, 760, and/or 790 (FIG. 7)) may include one or more monitoring components (e.g., monitoring component 316 (FIG. 3)) or monitoring sub-components, which may include an EMG. If control circuitry 304 determines to monitor the brain activity of a user using an EMG, control circuitry 304 proceeds to step 908, determines a measurement of electrical activity of a user of one or more regions of the brain of a user, and then proceeds to step 912. If control circuitry 304 determines not to use an EMG (e.g., an EMG is not included in the monitoring component), control circuitry 304 proceeds to step 910.

At step 910, control circuitry 304 determines whether or not to monitor the brain activity of a user using a default measuring technique. For example, the user device (e.g., user device 700, 730, 760, and/or 790 (FIG. 7)) may include one or more monitoring components (e.g., monitoring component 316 (FIG. 3)) or monitoring sub-components, which may be configured for functional magnetic resonance imaging (“fMRI”), which tracks brain activity by monitoring the levels of oxygenated blood that travels to active neurons, a positron emission tomography scan (“PET scan”), which tracks neurons' use of glucose in response to a stimulus, or any other suitable technique. If control circuitry 304 determines to monitor the brain activity of a user using a default measuring technique, control circuitry 304 determines a measurement of the brain activity of a user of one or more regions of the brain of a user using the technique, and proceeds to step 912. It should be noted that, in some embodiments, control circuitry 304 may obtain one or more of the measurements using one or more techniques for monitoring brain activity.

At step 912, control circuitry 304 retrieves a threshold range associated with a stimulus from a database. The threshold range is associated with the stimulus that is included in an advertisement displayed to the user. Depending on the type of monitoring used, the threshold range for the stimulus may be associated with the frequency range of the brain activity or the electrical activity of muscles near the brain. Control circuitry 304 may select a threshold range corresponding to the presented advertisement based on a time when the advertisement was displayed, a user input indicating the advertisement, metadata associated with the advertisement, or other suitable information. For example, a COKE advertisement may correspond to a particular threshold range while a FORD advertisement may correspond to a different threshold range.

In some embodiments, the database may be stored locally on storage 308 (FIG. 3) or stored remotely at media guidance data source 418 (FIG. 4) and/or any location accessible via communications network 414 (FIG. 4). Additionally or alternatively, the information in the database may be generated by a third party. For example, control circuitry 304 may receive data associated with an advertisement from a remote source (e.g., media content source 416 (FIG. 4)) that indicates a threshold range for a stimulus included in the advertisement.

At step 914, control circuitry 304 determines whether the determined measurement of brain activity matches the retrieved threshold range associated with the stimulus. For example, in some embodiments, the determined measure of brain activity is a brain state associated with a particular frequency range, electrical activity, and/or threshold range. These quantitative measurements may be compared by control circuitry 304 to determine whether or not the two measurements match (e.g., within a particular degree of deviation). For example, control circuitry 304 may determine whether the two measurements share the same frequency bands.

If control circuitry 304 determines that the measurements do correspond, control circuitry 304 proceeds to step 916 and returns a message indicating a match between the determined measure of brain activity and the retrieved threshold range. If control circuitry 304 determines that the measurements do not correspond (e.g., the brain state is different from the threshold range), control circuitry 304 proceeds to step 918 and returns a message indicating there was no match between the determined measure of brain activity and the retrieved threshold range.

It is contemplated that the steps or descriptions of FIG. 9 may be used with any other embodiment of this disclosure. In addition, the steps and descriptions described in relation to FIG. 9 may be done in alternative orders or in parallel to further the purposes of this disclosure. For example, each of these steps may be performed in any order or in parallel or substantially simultaneously to reduce lag or increase the speed of the system or method. Furthermore, it should be noted that any of the devices or equipment discussed in relation to FIGS. 3-4 and 7 could be used to perform one or more of the steps in FIG. 9.

The above-described embodiments of the present disclosure are presented for purposes of illustration and not of limitation, and the present disclosure is limited only by the claims which follow. Furthermore, it should be noted that the features and limitations described in any one embodiment may be applied to any other embodiment herein, and flowcharts or examples relating to one embodiment may be combined with any other embodiment in a suitable manner, done in different orders, or done in parallel. In addition, the systems and methods described herein may be performed in real-time. It should also be noted, the systems and/or methods described above may be applied to, or used in accordance with, other systems and/or methods.

1. A method for determining whether a viewer watched an advertisement, the method comprising:
generating for display an advertisement including a stimulus, wherein the stimulus is incorporated into the advertisement by a content provider in order to trigger a region of a brain of the viewer;
monitoring brain activity in the region of the brain of the viewer during the advertisement;
determining a brain state associated with the region based on the brain activity of the viewer during the advertisement;
comparing the brain state to a threshold range associated with the stimulus included in the advertisement; and
in response to determining that the brain state matches the threshold range, storing in a database an indication that the viewer watched the advertisement.

2. The method of claim 1, wherein the stimulus includes one or more of a visual stimulus, an audio stimulus, an audiovisual stimulus, a video stimulus, a graphical stimulus, and a textual stimulus.

3. The method of claim 1, wherein monitoring the brain activity of the viewer further comprises receiving data from an electroencephalogram unit indicating a frequency range of the brain activity of the viewer or receiving data from an electromyogram unit indicating electrical activity of muscles near the brain of the viewer at rest and during contraction, and wherein the threshold range is associated with the frequency range of the brain activity or the electrical activity of muscles near the brain.

4. The method of claim 1, wherein the region of the brain includes an occipital lobe, a parietal lobe, a temporal lobe, or a frontal lobe.

5. The method of claim 1, wherein the threshold range is associated with a response from the region of the brain in response to the stimulus included in the advertisement.

6. The method of claim 1, wherein the stimulus includes a strobe of light, the region includes an occipital lobe of the viewer, and the threshold range is associated with a response from the occipital lobe in response to the strobe of light displayed during the advertisement.

7. The method of claim 1, further comprising:
cross-referencing with the database to determine the advertisement the viewer has watched.

8. The method of claim 7, wherein the database includes a reference brain state associated with the advertisement, and wherein cross-referencing with the database to determine the advertisement the viewer has watched comprises determining that the brain state matches the reference brain state.

9. The method of claim 7, wherein the database includes reference metadata associated with the advertisement, and wherein cross-referencing with the database to determine the advertisement the viewer has watched comprises determining that metadata for the advertisement matches the reference metadata.

10. The method of claim 7, further comprising:
updating a count for a number of instances the viewer has watched the advertisement.

11. A system for determining whether a viewer watched an advertisement, the system comprising control circuitry configured to:
generate for display an advertisement including a stimulus, wherein the stimulus is incorporated into the advertisement by a content provider in order to trigger a region of a brain of the viewer;
monitor brain activity in the region of the brain of the viewer during the advertisement;
determine a brain state associated with the region based on the brain activity of the viewer during the advertisement;
compare the brain state to a threshold range associated with the stimulus included in the advertisement; and
in response to determining that the brain state matches the threshold range, store in a database an indication that the viewer watched the advertisement.

12. The system of claim 11, wherein the stimulus includes one or more of a visual stimulus, an audio stimulus, an audiovisual stimulus, a video stimulus, a graphical stimulus, and a textual stimulus.

13. The system of claim 11, wherein the control circuitry configured to monitor the brain activity of the viewer comprises the control circuitry configured to receive data from an electroencephalogram unit indicating a frequency range of the brain activity of the viewer or receive data from an electromyogram unit indicating electrical activity of muscles near the brain of the viewer at rest and during contraction, and wherein the threshold range is associated with the frequency range of the brain activity or the electrical activity of muscles near the brain.

14. The system of claim 11, wherein the region of the brain includes an occipital lobe, a parietal lobe, a temporal lobe, or a frontal lobe.

15. The system of claim 11, wherein the threshold range is associated with a response from the region of the brain in response to the stimulus included in the advertisement.

16. The system of claim 11, wherein the stimulus includes a strobe of light, the region includes an occipital lobe of the viewer, and the threshold range is associated with a response from the occipital lobe in response to the strobe of light displayed during the advertisement.

17. The system of claim 11, wherein the control circuitry is further configured to:
cross-reference with the database to determine the advertisement the viewer has watched.

18. The system of claim 17, wherein the database includes a reference brain state associated with the advertisement, and wherein the control circuitry configured to cross-reference with the database to determine the advertisement the viewer has watched comprises the control circuitry configured to determine that the brain state matches the reference brain state.

19. The system of claim 17, wherein the database includes reference metadata associated with the advertisement, and wherein the control circuitry configured to cross-reference with the database to determine the advertisement the viewer has watched comprises the control circuitry configured to determine that metadata for the advertisement matches the reference metadata.

20. The system of claim 17, wherein the control circuitry is further configured to:
update a count for a number of instances the viewer has watched the advertisement.

21-40. (canceled)
